(19) United States
(12) Patent Application Publication
(10) Pub. No.: US 2009/0035239 A1
(43) Pub. Date: Feb. 5, 2009

(54) Eye-shadow and process for its preparation

(75) Inventor: Piero Origo, Mozzanica (IT)

Correspondence Address: YOUNG & THOMPSON, 209 Madison Street, Suite 500, ALEXANDRIA, VA, 22314, US

(73) Assignee: ART COSMETICS S.R.L., MOZZANICA (IT)

(21) Appl. No.: 12/068,053

(22) Filed: Feb. 1, 2008

(30) Foreign Application Priority Data
Feb. 2, 2007 (IT) MI2007A172

(51) Int. Cl. A61K 8/92; A61Q 1/10

(52) U.S. Cl. 424/63

(57) ABSTRACT

New eye-shadow and process for the preparation thereof, characterised in that the conventional stage of preparation of the powder cake is followed by two further stages: an intermediate stage at which the cake is wetted with an oily compound, and a final stage at which the oily compound is absorbed.

The present invention relates to a new type of eye-shadow and the process for its preparation.

### INTRODUCTION

A large proportion of the global turnover of the cosmetics market is generated by eye make-up products, the most commonly used of which are eye-shadows.

There are different types of eye-shadow, which can be manufactured according to different technologies:
compact eye-shadows, prepared by a process that comprises grinding of powders (fillers and pigments) together with appropriate binder compounds, sieving of the powders to obtain a uniform particle size, and subsequent compacting under pressure to form a cake, known as the “compact”, which is placed in a metal container;
moulded eye-shadows, obtained by a process that comprises melting of waxes and oils in boilers, followed by the addition of a dispersion of suitably calendered pigments to the molten block; the block is then solidified into solid cakes which are finally re-melted and poured into the metal container, where they are left to solidify to obtain a cake known as the “moulding”;
“mousse” eye-shadows, prepared by mixing oils (mainly silicone oils), gelling agents and elastomers in boilers with pigments and viscosity-controlling agents, to obtain an aerated product with a soft, semisolid consistency which is inserted into the container under pressure to form a “mousse”.

The substantial difference between said three products lies in their organoleptic and application characteristics, namely the global sensory characteristics perceived by the user during use, normally described as “texture” in the cosmetics industry. The texture mainly comprises:
the appearance of the product, which is powdery and opaque in the case of compact eye-shadows, shiny and smooth in the case of moulded eye-shadows, and soft and “light” (because it is aerated) in the case of mousse eye-shadows;
the tactile sensation when the eye-shadow is taken up, which is dry and powdery in the case of compact eye-shadows, creamy and oily in the case of moulded eye-shadows, and soft in the case of mousse eye-shadows;
the tactile sensation during spreading, which is thick and dry in the case of compact eye-shadows, smooth and creamy in the case of moulded eye-shadows, and soft and rubbery in the case of mousse eye-shadows; and finally;
the shimmer, which depends substantially on the concentration of pearls (highly reflective mixtures based on mica, titanium dioxide, iron oxides, silica and colorants) used, and on the matrix in which they are dispersed; generally, pearl concentration being equal, moulded eye-shadows are brightest, compact eye-shadows are less bright, and mousse eye-shadows are still less bright.

The various textures present different degrees of “persistence” on the skin over time: long persistence is defined as the “long-lasting” effect.

### DESCRIPTION OF THE INVENTION

The present invention relates to a new type of eye-shadow, characterised by a unique sensory perception, with a texture that includes various characteristics which have never before coexisted in a single formula, but are usually associated with different types of products, and the process for its preparation.

The eye-shadow according to the present invention has the same appearance as a compact eye-shadow, but it presents characteristics similar to those of mousse eye-shadows when it is taken up, and similar to those of cast eye-shadows when it is spread, and also presents a unique, exceptional shimmer because the characteristics of the pearls it contains are enhanced to a much greater extent than in conventional eye-shadows.

More particularly, the eye-shadow according to the invention presents the following characteristics:
compact product appearance;
texture similar to that of a mousse when the product is taken up;
texture on spreading very soft and creamy, similar to that of a moulded product;
a more persistent texture;
greater shimmer and enhanced characteristics of the pearls.

The present invention also relates to the process for the preparation of said eye-shadow, characterised in that the conventional preparation stage of the powder cake is followed by two further stages: an intermediate stage at which the cake is wet with an oily compound, and a final stage at which the oily compound is absorbed.

More particularly, the process to which the invention relates comprises:

A) a stage at which the powder cake is prepared by mixing the constituents of the eye-shadow, such as colorants, talc, mica, silica, pearls and binder, in a mill; sieving the powder obtained, and finally, compacting the powder, which is distributed between appropriate metal containers;

B) a stage at which the cake is wetted with an oily compound, which may be a fatty alcohol, an ester, a silicone oil, a mineral oil, a vegetable oil or a triglyceride, which is poured onto the compacted cake through a hopper;

C) a final stage at which the oily compound is slowly absorbed into the interstices of the compacted cake, which first swells and then gradually returns to its original shape.

The various stages of the process according to the invention are described in greater detail below.

A1. Preparation of the Powder Cake (Bulk)

In the jargon of the cosmetics industry, the term “bulk” refers to the semi-manufactured product, in liquid or solid form, which is to be transferred, with or without heating, to the final container.

The components listed below, which are normally found in the composition of an eye-shadow, are mixed in a mill of suitable dimensions fitted with a mixing blade, in the following percentages in weight of the end product:
colorants 1-5% (preferably around 2-3%)
talc 5-30% (preferably around 15-18%)
mica 5-30% (preferably around 15-18%)
silica 0-5% (preferably around 1-2%)
pearls 20-60% (preferably 40-45%)
binder (mixture of oils and gel) 10-50% (preferably 20-30%).

The mixture is ground for a time ranging between 2 and 15 minutes (preferably around 5 min).

According to the invention, the colorants will be chosen from the group of synthetic organic compounds, generally azo compounds, and inorganic compounds such as transition metal oxides (e.g. FeO and Fe₂O₃) of various colours.

According to the invention, the pearls can be highly reflective mixtures, mainly containing mica and titanium dioxide (TiO₂) in variable proportions, amounting to over 80%, and smaller percentages of ferrous iron oxides (FeO) and ferric iron oxides (Fe₂O₃), silica and various colorants.

According to the invention, the binders will be chosen from among mixtures of oils, including liquid paraffin, triglycerides, esters and alcohols with a long chain of carbon atoms (C10-C22), silicone oils and/or gels obtained by polymerisation and copolymerisation of unsaturated aliphatic monomers, also with a long chain of carbon atoms.

A2. Sieving

The powder is sieved in a suitable sieving machine (preferably a vibrating sieve) for a time ranging between 10 and 60 minutes (preferably 30 min).

A3. Compacting

The powder is distributed between metal containers (generally made of aluminium or tinplate) commonly called “trays”, with a variable shape (generally circular or quadrilateral) and highly variable dimensions (from 10 to 100 mm), and subjected to a pressing stage in suitable machines with a press, from which the powder is separated by a thin layer of cloth.

The pressing operation may be performed once only or repeated two or three times, and is normally performed at a pressure ranging between 10 and 80 atm (preferably 20 or 30 atm).

The trays containing the cakes of pressed powder are dusted with compressed air and packaged.

B. Wetting

The compacted cake then undergoes a wetting stage instead of being packaged immediately in its sales packaging (e.g. a make-up case), as is traditionally done.

The wetting agent is generally an oily compound such as:
a saturated or unsaturated fatty alcohol;
a saturated or unsaturated ester with a medium or long chain;
a silicone oil;
a mineral oil;
a vegetable oil;
a triglyceride.

The oil is poured through a suitable hopper onto the compacted cake by a nozzle with a diameter ranging between 1 mm and 1 cm (preferably around 5 mm) so that it forms a “drop” on the cake.

The amount of oil absorbed by the cake is proportional to the weight of the cake. A quantity ranging between 10% and 30% in weight of the compacted powder is used.

C. Absorption Stage

The oil is left to be absorbed slowly into the interstices of the compacted cake for a time ranging between 10 and 30 hours (preferably between 12 and 16 hours), possibly at ambient temperature.

The cake first swells, and then gradually returns to its original shape. It takes on a soft surface appearance that enhances the presence of the pearls, modifying the traditional application characteristics and acquiring the performances indicate above, which are characteristic of the eye-shadow according to the invention.

1. Process for the preparation of eye-shadow, characterised in that the conventional stage of preparation of the cake in powder form is followed by two further stages: an intermediate stage at which the cake is wetted with an oily compound, and a final stage at which the oily compound is absorbed.

2. Process as claimed in claim 1, characterised in that it includes the following stages:
a) preparation of the powder cake by mixing the components of the eye-shadow in a mill, sieving the powder obtained, and compacting the powder which has been distributed between suitable metal containers;
b) wetting of the cake with an oily compound;
c) slow absorption of the oily compound into the compacted cake.
d)

3. Process as claimed in claim 2, characterised in that the constituents listed below are mixed at stage a) in the following percentages in weight of the end product:
colorants 1-5%, preferably 2-3%;
talc 5-30%, preferably 15-18%;
mica 5-30%, preferably 15-18%;
silica 0-5%, preferably 1-2%;
pearls 20-60%, preferably 40-45%;
binder (mixture of oils and gels) 10-50%, preferably 20-30%.

4. Process as claimed in claim 3, characterised in that the oily compound used at stage b) is chosen from the group of fatty alcohols, esters, silicone oils, mineral oils, vegetable oils and triglycerides.

5. Process as claimed in claim 4, characterised in that the oily compound used at stage b) is chosen from among liquid paraffin, triglycerides, esters and alcohols with a long chain of carbon atoms (C10-C22), silicone oils and/or gels obtained by polymerisation and copolymerisation of unsaturated aliphatic monomers with a long chain of carbon atoms, or mixtures thereof.

6. Process as claimed in claim 1, characterised in that the oily compound used at stage b) is added in a quantity ranging between 10% and 30% of the weight of the compacted powder.

7. Process as claimed in claim 1, characterised in that the oily compound used at stage b) is poured through a nozzle with a diameter ranging between 1 mm and 1 cm, preferably around 5 mm.

8. Process as claimed in claim 1, characterised in that the oily compound used at stage b) is left to be absorbed slowly into the interstices of the compacted cake for a time ranging between 10 and 30 hours, preferably between 12 and 16 hours.

9. Eye-shadow obtainable by the process described in claim 1.

10. Process as claimed in claim 2, characterised in that the oily compound used at stage b) is added in a quantity ranging between 10% and 30% of the weight of the compacted powder.

11. Process as claimed in claim 2, characterised in that the oily compound used at stage b) is poured through a nozzle with a diameter ranging between 1 mm and 1 cm, preferably around 5 mm.

12. Process as claimed in claim 2, characterised in that the oily compound used at stage b) is left to be absorbed slowly into the interstices of the compacted cake for a time ranging between and 30 hours, preferably between 12 and 16 hours.
